(19) United States
(12) Patent Application Publication
(10) Pub. No.: US 2005/0261696 A1
(43) Pub. Date: Nov. 24, 2005

(54) Apparatus for setting a cutting block for a resection of the tibia

(75) Inventors: Tom Overes, Wintherthur (CH); Susanne Fischli, Buelach (CH); Francisco Faoro, Zuerich (CH); Thomas Trachsler, Winterthur (CH)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Zimmer GmbH, Winterthur (CH)

(21) Appl. No.: 11/125,650

(22) Filed: May 9, 2005

(30) Foreign Application Priority Data
May 17, 2004 (EP) 04011687.3

(51) Int. Cl. A61F 5/00

(52) U.S. Cl. 606/87

(57) ABSTRACT

The invention relates to an apparatus for setting a cutting block having at least one guide surface for a resection of the tibia, the apparatus being capable of being anchored to the tibia in both an extramedullary and an intramedullary manner, and comprising a column which can be aligned with respect to the tibia and which is connected to a transverse arm provided for the anchoring at the natural tibia platform, with the cutting block being vertically adjustable relative to the transverse arm, wherein a carrier device which is vertically adjustable at the column is provided for the cutting block and has a joint arrangement by which different inclinations of the guide surface of the cutting block toward lateral/medial and/or toward posterior can be set.

The invention relates to an apparatus for setting a cutting block having at least one guide surface for a resection of the tibia, the apparatus being capable of being anchored to the tibia in both an extramedullary and an intramedullary manner, and comprising a column which can be aligned with respect to the tibia and which is connected to a transverse arm provided for anchoring at the natural tibia platform, with the cutting block being vertically adjustable relative to the transverse arm.

Such an apparatus for the setting of a cutting block for a resection of the tibia is generally known. The apparatus serves in particular for the alignment of the guide surface of the cutting block at the tibia, with the aligned cutting block being secured to the tibia by means of pins. A saw blade guide can subsequently be set onto the cutting block and the resection of the tibia can be carried out by means of a saw blade. The known apparatus can be anchored to the tibia via the transverse arm both in an extramedullary manner outside the medullary space and in an intramedullary manner using a medullary nail set along the tibia axis.

With an extramedullary anchoring, the column connected to the transverse arm substantially at right angles can be extended by means of an extension rod and can be supported on the tibia by means of a compound rest in the region of the ankle, with a correction of the inclination of the guide surface of the cutting block with respect to the transverse axis or the sagittal axis being able to be achieved by means of the compound rest which can be displaced along the transverse axis and the sagittal axis. With an intramedullary anchoring, the apparatus can be rotated around the longitudinal axis of the medullary nail or around the tibia axis. After the correct alignment, the apparatus, in particular its transverse arm, is fixed to the tibia by hammering in one or more pins.

In the known apparatus, the cutting block is vertically adjustable along a straight-line guide connected to the transverse arm and extending parallel to the longitudinal axis of the column. The cutting block is held at the respective height via a support surface of a regulating nut which is secured to the column and can be adjusted along it.

It is, however, disadvantageous with such apparatuses for the setting of a cutting block having at least one guide surface for a resection of the tibia that, with an extramedullary anchoring, the alignment of the apparatus can only be carried out by means of an additional compound rest and, with an intramedullary anchoring, the apparatus can only be rotated around the longitudinal axis of the medullary nail.

An apparatus is moreover also known which permits a pivoting of the total straight-line guide (along which the cutting block is vertically adjustable) around the sagittal axis and the transverse axis. With such an apparatus, the transverse arm is made in two parts, with the two components being pivotal with respect to one another around a pivot axis. A further pivot axis is furthermore provided between the straight-line guide and the transverse arm. It is, however, disadvantageous here that the apparatus has a relatively complex design and is awkward to handle, with elements to be actuated to carry out the pivot movements in particular being arranged to posterior of the column and thus close to the operation aperture.

It is the underlying object of the invention to provide an apparatus of the initially named kind which has a space-saving and simultaneously simple design and can be aligned as simply as possible with respect to the tibia to be resected, with in particular a correction of a defective position of the legs being made possible.

This object is satisfied by the features of claim 1 and in particular in that a carrier device which is vertically adjustable at the column is provided for the cutting block and has a joint arrangement by which different inclinations of the guide surface of the cutting block toward lateral/medial and/or toward posterior can be set.

The invention is in particular characterized in that the carrier device satisfies a dual function since it is configured, on the one hand, for the vertical adjustment of the cutting block and is simultaneously configured for the correction of the inclination of the guide surface of the cutting block toward lateral/medial and/or toward posterior. The vertical adjustment of the cutting block can be achieved by adjustment of the carrier device at the column and the correction of the inclination of the guide surface of the closing block can be achieved by the joint arrangement of the carrier device. A particularly compact and thus space-saving design of the apparatus in accordance with the invention can hereby be achieved.

A major advantage of the invention lies in the fact that, with an extramedullary anchoring, the alignment of the apparatus for the setting of a cutting block can also be carried out without making use of an additional compound rest and, with an intramedullary anchoring, a correction of the inclination of the guide surface of the cutting block with respect to the transverse axis or the sagittal axis can also be achieved.

Advantageous embodiments of the invention are recited in the dependent claims, in the description and in the drawing.

It is furthermore proposed in accordance with the invention that the carrier device has a carrier arm which is arranged transversely to the column to hold the cutting block and whose both ends preferably project beyond the column in a radial direction. It is hereby in particular possible to carry out the operation in a manner which is as minimally invasive as possible. The cutting block can in particular be placed onto the one end of the carrier arm or can be held by this in another manner. The cutting block and the carrier arm are preferably adjusted together by the joint arrangement for the correction of the inclination of the guide surface of the cutting block.

The joint arrangement preferably includes at least two pivot axes, in particular oriented perpendicular to one another. The one pivot axis is preferably oriented in the anterior/posterior direction to permit a correction of the inclination of the guide surface of the closing block toward lateral/medial and thus a correction of a varus/valgus position of the legs of a patient, whereas the other pivot axis is oriented in the medio-lateral direction to permit a correction of the inclination of the guide surface of the cutting block toward posterior.

It is particularly advantageous for the end of the carrier device carrying the cutting block to be arranged to posterior of the column and for the joint arrangement to be arranged to anterior of the column. It can hereby be ensured that elements of the joint arrangement and/or carrier device for the carrying out of the correction of the inclination of the guide surface of the cutting block are easily accessible for the surgeon and are as far away as possible from the operation aperture.

Furthermore, in accordance with the invention, the carrier arm can be adjustable in the anterior/posterior direction. It is hereby achieved that the cutting block held by the carrier arm is placed onto the carrier arm in a position of the carrier arm displaced toward anterior and can subsequently be moved toward the tibia by a displacement of the carrier arm in the posterior direction. The correction of the inclination of the guide surface of the closing block can be carried out in any desired position of the carrier arm adjustable in the anterior/posterior direction. The carrier arm and/or the carrier device can in particular be provided with a ratchet device which only permits discrete positions of the carrier arm in the anterior/posterior direction or with a stopping device to stop the carrier arm in a specific position in the anterior/posterior direction.

In accordance with an advantageous embodiment of the invention, the column has a straight-line guide for the vertical adjustment of the carrier device which cooperates with a slide device of the carrier device. The vertical adjustment of the carrier device at the column can be realized in a particularly simple manner by means of the straight-line guide and the slide device. The carrier device is held at the column in a slidingly displaceable manner.

The column is preferably configured for the making of the straight-line guide as a tube, in particular having a circular or a rectangular inner cross-section. The straight-line guide can hereby be realized in a particularly simple manner. Alternatively, the straight-line guide can, however, also be arranged along a longitudinal axis of the column, for example, as a slider guide or as a rail on an outer side of a cylindrical basic shape of the column.

It is furthermore preferred for the slide device to include at least two slide elements which are spaced apart from one another in the direction of a longitudinal axis of the column and are in particular made as sliding blocks. The two slide elements are designed to cooperate with the straight-line guide of the column and are each supported in the straight-line guide of the column in a slidingly displaceable manner. The carrier arm can in particular be arranged between the two slide elements. A particularly stable design of the carrier device thereby results.

The slide elements are preferably spherical in shape for support in a tube which in particular has a circular inner cross-section. The slide elements are preferably cylindrical in shape for support in a tube which in particular has a rectangular inner cross-section. Generally, however, any desired design of the inner cross-sections of the tube and any desired shapes of the slide elements are possible.

In accordance with a preferred embodiment of the invention, a base body of the carrier device is pivotable relative to the column about a first pivot axis oriented in the medio-lateral direction for the correction of the inclination of the guide surface of the cutting block with respect to the sagittal axis. The carrier arm with the base body is preferably pivotable around the first pivot axis.

At least one slide element can furthermore be made for the vertical adjustment of the carrier device and simultaneously for the pivoting of the base body of the carrier device. The slide element can be supported in a slidingly displaceable manner along a straight-line guide of the column and can simultaneously act as part of a rotary mechanism for the base body, with the translatory movement of the carrier device and the pivot movement of the base body being able to be superimposed on one another.

In accordance with a particularly preferred embodiment, the spacing of at least one slide element from the base body can be varied for the pivoting of the base body. With two slide elements spaced apart from one another in the direction of a longitudinal axis of the column, the base body can be pivoted or tilted with respect to the column by the change of the spacing of one slide element from the base body.

To permit a pivot movement or tilt movement of the base body with respect to the column, the spacing of the other slide element to the base body is preferably kept constant. Alternatively, the spacing of the other slide element to the base body can, however, also be changed in an opposing manner to the spacing of the one slide element to the base body or analog to the spacing of the one slide element to the base body, but with a different increment.

The slide element and the base body are preferably connected to one another via a regulating screw connection to change the spacing between the slide element and the base body. At least one of these two components, slide element and/or base body, can be provided with a thread of the regulating screw connection such that the spacing between the slide element and the base body is changed on a turning of the screw.

The slide elements are preferably each connected to a base body via a connection element, in particular via an elongated connection element, with preferably at least one connection element being provided with a regulating thread of a regulating screw connection. The regulating screw connection is formed by the regulating thread of the connection element and by a thread of the slide element and/or of the base body. The connection elements are preferably oriented parallel to a carrier arm of the carrier device.

In accordance with a particular embodiment of the invention, the column is slotted at least regionally, with the carrier arm in particular being guided between two slide elements through the slot formed in the column. The apparatus for setting a cutting block can hereby be made in a particularly space-saving and compact manner. Alternatively, an apparatus is, however, also feasible in which the column is not slotted and the carrier arm is guided around the column on one or two sides.

To secure the carrier arm against rotation with respect to a rotation around a longitudinal axis of the column, preferably at least one securing element of the carrier device is in engagement with the slot. The end of the securing element remote from the carrier device can be arranged inside the column or project out of it on the side of the column disposed opposite the carrier device. The securing element can be attached to a base body of the carrier device and/or to a slide element of the carrier device.

In accordance with a further preferred embodiment of the invention, a holder element of the carrier device is pivotable about a second pivot axis oriented in the medio-lateral direction for the correction of the inclination of the guide surface of the cutting block with respect to the transverse axis. The carrier arm with the holder element is preferably pivotable around the second pivot axis. The holder element is in particular made as a driver for the carrier arm. To pivot the holder element, a threaded rod adjustable in the medio-lateral direction is preferably provided into which the holder element engages.

The inclination of the guide surface of the cutting block with respect to lateral/medial and/or with respect to posterior is particularly easily recognizable when the angle of a pivot movement about a first and/or second axis can respectively be read off a scale. The correction angles determined by X-rays in the operation planning can hereby in particular be set simply and reliably.

The invention will be described in the following by way of example with reference to the drawing. There are shown:

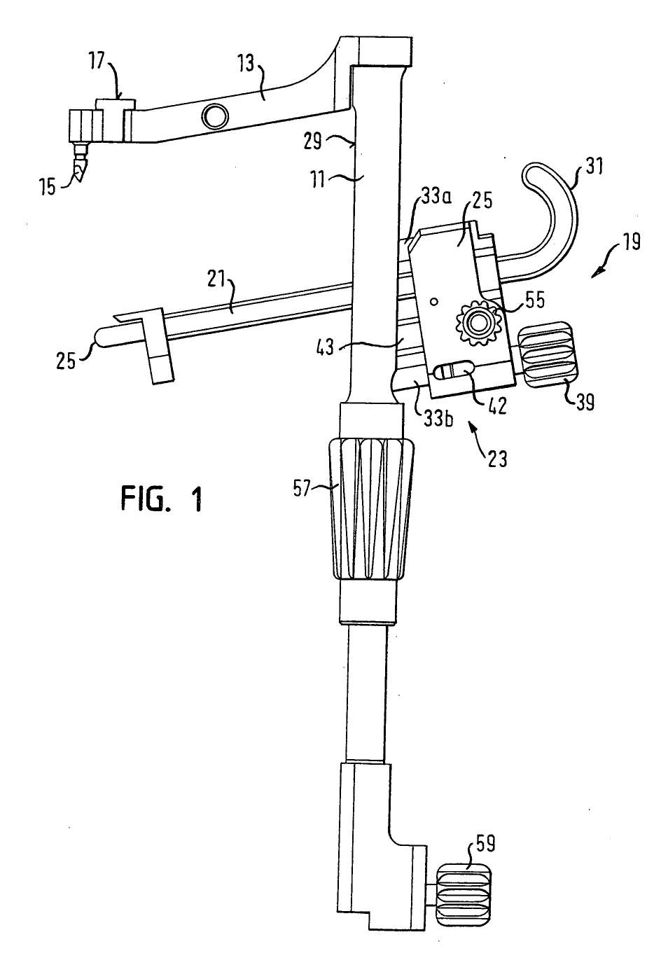

FIG. 1 a side view of an apparatus in accordance with the invention for the setting of a cutting block;

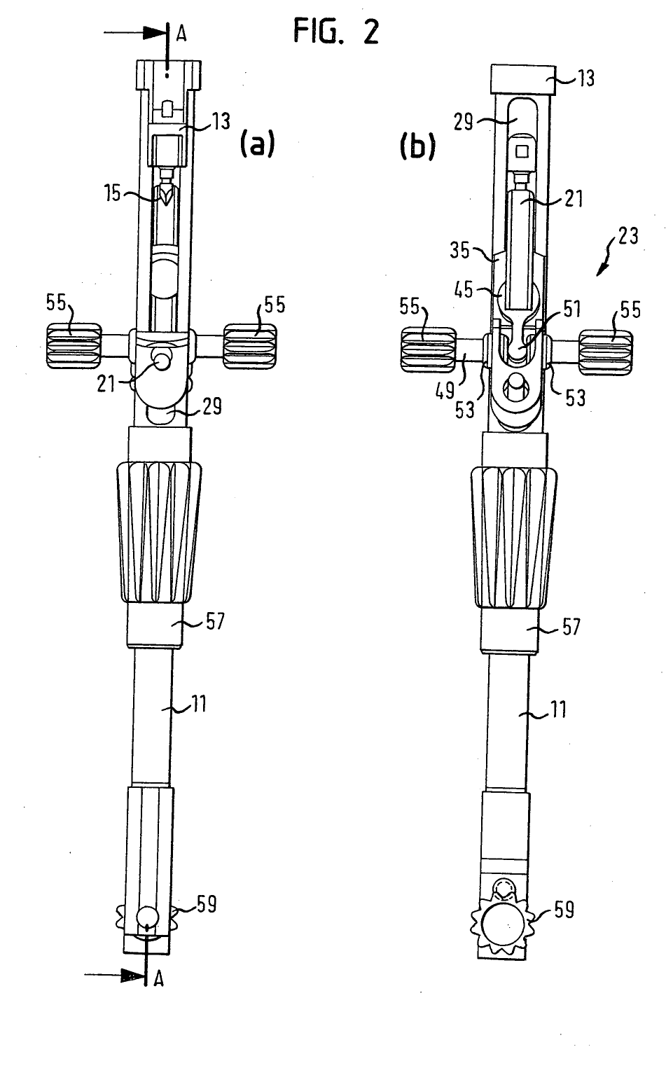

FIG. 2a, b views of the apparatus of FIG. 1 from posterior and anterior respectively;

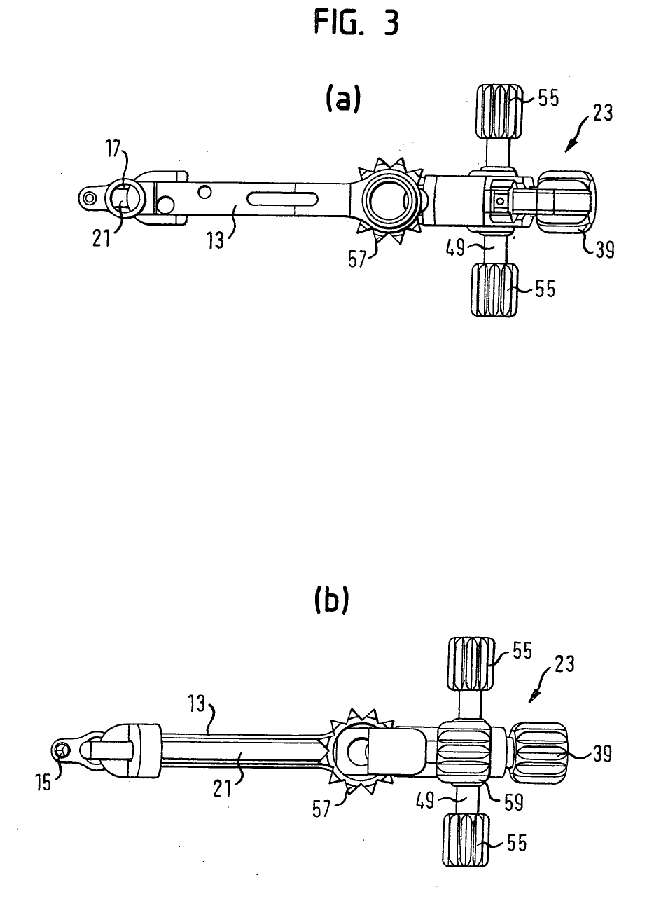

FIG. 3a, b views of the apparatus of FIG. 1 from proximal and distal respectively;

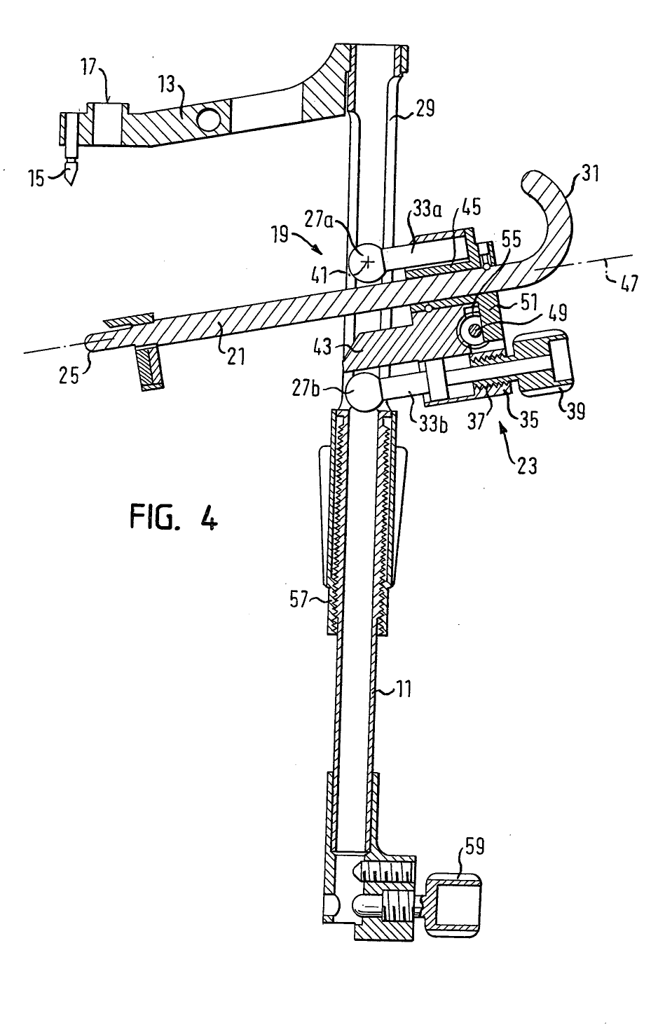

FIG. 4 a longitudinally sectioned side view of the apparatus of FIG. 1 along the line A-A in FIG. 2a; and

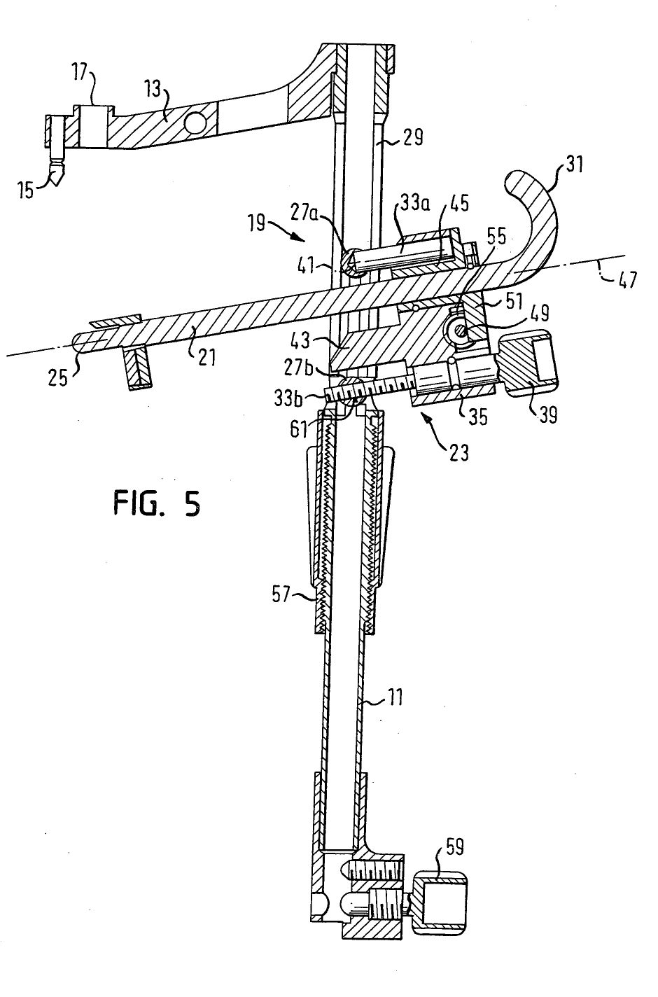

FIG. 5 a longitudinally sectioned side view of a further embodiment of the apparatus in accordance with the invention.

FIGS. 1 to 3 show an apparatus in accordance with the invention for the setting of a cutting block (not shown) having guide surfaces for a resection of a tibia (not shown) from different directions. The apparatus includes a column 11 which can be aligned with respect to the tibia and a transverse arm 13 which is connected substantially at right angles to the column 11 and which can be anchored to the natural tibia platform in either an extramedullary or intramedullary manner. With an extramedullary anchoring, a pin 15 projecting from the transverse arm 13 toward distal is set. With an intramedullary anchoring, a medullary nail (not shown) is additionally set which is hammered into the tibia along a guide bore 17 formed in the transverse arm 13.

The setting apparatus in accordance with the invention furthermore includes a carrier device 19 which is vertically adjustable at the column 11 along a longitudinal axis of the column 11. The carrier device 19 has a carrier arm 21 arranged transversely to the column 11 for the holding of the cutting block and a joint arrangement 23 which permits the setting of different inclinations of the guide surfaces of the cutting block held by the carrier arm 21 toward lateral/medial and toward posterior. The end 25 of the carrier arm 21 carrying the cutting block is arranged to posterior and the joint arrangement 23 is arranged to anterior of the column 11.

In accordance with FIG. 4, the column 11 is formed as a tube with a circular inner cross-section. The tube 11 forms a straight-line guide which is oriented in the longitudinal direction of the column 11 and into which two spherical groove blocks 27 of the joint arrangement 23 spaced apart from one another in the longitudinal direction of the column 11 are slidingly displaceably inserted. The two groove blocks 27 form a slide device of the joint arrangement 23 by means of which the carrier device 19, i.e. the joint arrangement 23 and the carrier arm 21, are vertically adjustable.

The carrier arm 21, which is additionally adjustable in the anterior/posterior direction, is guided between the two groove blocks 27 through a slot 29 formed in the column 11, penetrating the column 11 from anterior to posterior and extending in the longitudinal direction of the column 11, as can in particular be seen in FIG. 2. The slot 29 passes through both the wall facing to anterior and the wall facing to posterior of the tubular column 11. The end 31 of the carrier arm 21 opposite the end 25 carrying the cutting block is curved and made in the form of an engagement depression into which the surgeon can engage easily with one finger to adjust the carrier arm 21 in the anterior/posterior direction.

The groove blocks 27 are connected to a base body 35 of the joint arrangement 23 via a respective elongated connection element 33, with the connection elements 33 being guided through the slot 29. The two connection elements 33 are firmly connected to the respective groove block 27. The upper connection element 33a associated with the upper groove block 27a arranged above the carrier arm 21 is still firmly connected to the base body 35.

The lower connection element 33b associated with the lower groove block 27b arranged beneath the carrier arm 21 is, in contrast, connected to the base body 35 via a regulating screw connection 37. To form the regulating screw connection 37, the connection element 33b is provided with a regulating thread in the form of an external thread and the base body 35 is provided with a corresponding internal thread. The spacing between the lower groove block 27b and the base body 35 can be varied by means of the regulating screw connection 37. A rotary knob 39, which is not shown in FIG. 2b for reasons of clarity, is provided for the adjustment of the regulating screw connection 37.

The base body 35 is pivotable relative to the column 11 about a first pivot axis 41 oriented in the medio-lateral direction and extending perpendicular to the plane of the drawing in FIG. 4 for the correction of the inclination of the guide surfaces of the cutting block with respect to the sagittal axis. A pivot movement about the first pivot axis 41 can be effected by rotating the rotary knob 39, i.e. by changing the spacing between the lower groove block 27b and the base body 35, since the spacing between the upper groove block 27a and the base body 35 is fixed. The lower groove block 27b is thus made both for the vertical adjustment of the carrier device 19 and for the pivoting of the base body 35. The angle of the pivot movement of the base body 35 about the first pivot axis 41 can be read off a scale 42 which is shown in FIG. 1.

For the rotational securing of the carrier arm 21 or of the carrier device 19 about the longitudinal axis of the column 11, a securing element 43 is provided whose at least one dimension is matched to the width of the slot 29 such that the securing element 43 is in engagement with the slot 29 of the column 11 at least approximately free of play. The securing element 43 is attached to the base body 35 and is arranged between the carrier arm 21 and the lower groove block 27b.

For the correction of the inclination of the carrier arm 21 with respect to the transverse axis, a holder element 45 of the carrier device 19 is pivotable about a second pivot axis 47 which is oriented in the anterior/posterior direction and which extends perpendicular to the first pivot axis 41. In a plane extending perpendicular to the second pivot axis 47, the carrier arm 21 has a rectangular outer cross-section which is matched to a rectangular inner cross-section of the holder element 45, as can be recognized in FIG. 2b. The holder element 45 is in particular made as a driver for the carrier arm 21.

To pivot the holder element 45, a threaded rod 49 adjustable in the medio-lateral direction is provided into which a clapper 51 of the holder element 45 in particular shown in FIG. 2b engages. The clapper 51 is arranged between a first and a second external thread section 53 of the threaded rod 49. The external thread sections 53 form a further regulating screw connection with corresponding internal thread sections (not shown) of the base body 35. The region of the threaded rod 49 which is formed between the two external thread sections 53 and into which the clapper 51 engages has no thread.

A respective rotary knob 55 is attached to each of the two ends of the threaded rod 49. The threaded rod 49 can be adjusted in the medio-lateral direction by rotating one of the two rotary knobs 55. The two external thread sections 53 and the region which is formed between the two external thread sections 53 and into which the clapper 51 engages, and the clapper 51 are also correspondingly displaced. With the displacement of the clapper 51 in the medio-lateral direction, the holder element 47, which is rotationally movably supported in the base body, and thus the carrier arm 21, are pivoted about the second pivot axis 47.

The adjustment movement of the threaded rod 49 in the medio-lateral direction is restricted on both sides such that the carrier arm 21 can only be pivoted about the second pivot axis 47 up to a specific angular position and in a counter-clockwise direction. In particular an abutment of a transversely positioned carrier arm 21 at the boundaries of the slot 29 can hereby be prevented.

Any play which may be present between the holder element 47 and the carrier arm 21 can be eliminated, for example, by a spring-loaded pressure element of the one element 21, 47 which is pressed into a prismatic counter-surface of the other element 47, 21 or by any other measures which reduce play.

For the vertical adjustment of the carrier device 19, a regulating nut 57 is provided which is secured to the column 11 and which is adjustable along the longitudinal direction of the column 11 by means of an internal thread which engages into an external thread of the column 11. The carrier device 19 is held by the regulating nut 57 at the respective height adopted by the regulating nut 57. The external thread of the column 11 also extends to the region of the column 11 having the slot 29.

For the additional support of the apparatus in accordance with the invention with an extramedullary anchoring, an extension rod (not shown) can be introduced at the lower end of the column 11 and is supported on the tibia via a compound rest (not shown) in the region of the ankle. A clamping screw 59 attached to the column 11 is provided for the securing of the extension bar.

FIG. 5 shows a further embodiment of a setting apparatus in accordance with the invention, with respectively the same or corresponding parts being designated with the same reference numerals so that a repeat representation is dispensed with and only the differences of the embodiment shown in these Figures with respect to the first embodiment are explained.

In contrast to the embodiment in accordance with FIGS. 1 to 4, in the apparatus shown in FIG. 5, the lower connection element 33b and the base body 35 are not connected to one another by means of a regulating screw connection, but are firmly connected to one another. To permit a change in the spacing of the lower groove block 27b to the base body 35, in the further embodiment in accordance with FIG. 5, the lower groove block 27b and the lower connection element 33b are connected to one another by means of a regulating screw connection 61. A pivot movement of the base body 35 about the first pivot axis 41 can be effected by means of the rotary knob 39 analogously to the embodiment shown in FIGS. 1 to 4.

The apparatus in accordance with the invention explained with reference to the two embodiments for the setting of a cutting block includes a compact joint arrangement which is, on the one hand, adjustable vertically at the column of the apparatus together with the carrier arm and, on the other hand, permits a correction of the inclination of the guide surfaces of a cutting block toward lateral/medial and posterior. Despite the dual functionality, the joint arrangement is extremely space-saving and can be operated particularly easily by the surgeon.

### Reference Numeral List

11 column
13 transverse arm
15 pin
17 guide bore
19 carrier device
21 carrier arm
23 joint arrangement
25 end of the carrier arm
27 groove block
29 slot
31 end of the carrier arm
33 connection element
35 base body
37 regulating screw connection
39 rotary knob
41 pivot axis
42 scale
43 securing element
45 holder element
47 pivot axis
49 threaded rod
51 clapper
53 threaded section
55 rotary knob
57 regulating nut
59 clamping screw
61 regulating screw connection

1. An apparatus for setting a cutting block having at least one guide surface for a resection of the tibia, the apparatus being capable of being anchored to the tibia in both an extramedullary and an intramedullary manner, and comprising a column (11) which can be aligned with respect to the tibia and which is connected to a transverse arm (13) provided for the anchoring at the natural tibia platform, with the cutting block being vertically adjustable relative to the transverse arm (13), characterized in that a carrier device (19) which is vertically adjustable at the column (11) is provided for the cutting block and has a joint arrangement (23) by which different inclinations of the guide surface of the cutting block toward lateral/medial and/or toward posterior can be set.

2. An apparatus in accordance with claim 1, characterized in that the carrier device (19) for the holding of the cutting block has a carrier arm (21) arranged transversely to the column (11).

3. An apparatus in accordance with claim 1, characterized in that the joint arrangement (23) includes at least two pivot axes (41, 47) in particular oriented perpendicular to one another.

4. An apparatus in accordance with claim 1, characterized in that the end (25) of the carrier device (19) carrying the cutting block is arranged to posterior of the column (11) and the joint arrangement (23) is arranged to anterior of the column (11).

5. An apparatus in accordance with claim 2, characterized in that the carrier arm (21) is adjustable in the anterior/posterior direction.

6. An apparatus in accordance with claim 1, characterized in that the column (11) has a straight-line guide for the vertical adjustment of the carrier device (19) which cooperates with a slide device (27) of the carrier device (19).

7. An apparatus in accordance with claim 6, characterized in that the column is configured for the making of the straight-line guide as a tube (11), in particular having a circular or a rectangular inner cross-section.

8. An apparatus in accordance with claim 6, characterized in that the slide device includes at least two slide elements (27) which are spaced apart from one another in the direction of a longitudinal axis of the column (11) and are in particular made as groove blocks.

9. An apparatus in accordance with claim 8, characterized in that the slide elements (27) are spherical for the support in a tube (11) in particular having a circular internal cross-section.

10. An apparatus in accordance with claim 8, characterized in that the slide elements (27) are cylindrical for the support in a tube (11) in particular having a rectangular internal cross-section.

11. An apparatus in accordance with claim 1, characterized in that a base body (35) of the carrier device (19) is pivotable relative to the column (11) about a first pivot axis (41) oriented in the medio-lateral direction for the correction of the inclination of the guide surface of the cutting block with respect to the sagittal axis.

12. An apparatus in accordance with claim 11, characterized in that at least one slide element (27b) is made for the vertical adjustment of the carrier device (19) and simultaneously for the pivoting of the base body (35) of the carrier device (19).

13. An apparatus in accordance with claim 11, characterized in that the spacing of at least one slide element (27b) from the base body (35) can be varied for the pivoting of the base body (35).

14. An apparatus in accordance with claim 13, characterized in that the slide element (27b) and the base body (35) are connected to one another via a regulating screw connection (37).

15. An apparatus in accordance with claim 8, characterized in that the slide elements (27) are each connected to a base body (35) via a connection element (33), in particular via an elongated connection element, with preferably at least one connection element (33b) being provided with a regulating thread of a regulating screw connection (37).

16. An apparatus in accordance with claim 2, characterized in that the column (11) is slotted at least regionally, with the carrier arm (21) in particular being guided between two slide elements (27) through the slot (29) formed in the column (11).

17. An apparatus in accordance with claim 16, characterized in that at least one securing element (43) of the carrier device (19) is in engagement with the slot (29) for the rotational security of the carrier arm (21) with respect to a rotation about a longitudinal axis of the column (11).

18. An apparatus in accordance with claim 17, characterized in that the securing element (43) is attached to a base body (35) of the carrier device (19).

19. An apparatus in accordance with claim 17, characterized in that the securing element (43) is attached to a slide element (27) of the carrier device (19).

20. An apparatus in accordance with claim 1, characterized in that a holder element (45) of the carrier device (19) is pivotable about a second pivot axis (47) oriented in the anterior/posterior direction for the correction of the inclination of the guide surface of the cutting block with respect to the transverse axis.

21. An apparatus in accordance with claim 20, characterized in that the holder element (45) is made as a driver for the carrier arm (21).

22. An apparatus in accordance with claim 21, characterized in that, to pivot the holder element (45), a threaded rod (49) adjustable in the medio-lateral direction is provided into which the holder element (45) engages.

23. An apparatus in accordance with claim 1, characterized in that the angle of a pivot movement about a first and/or second axis (41, 47) can be read off at a scale (42).
